(19) United States
(12) Patent Application Publication
(10) Pub. No.: US 2017/0089894 A1
(43) Pub. Date: Mar. 30, 2017

(54) SYSTEMS AND METHODS FOR POINT-OF-CARE DETERMINATION OF LDL-C AND LDL-P

(71) Applicant: Polymer Technology Systems, Inc., Indianapolis, IN (US)

(72) Inventors: Aniruddha Patwardhan, Fishers, IN (US); Gary L. Hughes, Camby, IN (US)

(21) Appl. No.: 15/277,510

(22) Filed: Sep. 27, 2016

Related U.S. Application Data
(60) Provisional application No. 62/234,527, filed on Sep. 29, 2015.

(51) Int. Cl. G01N 33/558; G01N 33/92

(52) U.S. Cl. CPC G01N 33/558; G01N 33/92

(57) ABSTRACT

A system for testing for LDL-C and LDL-P includes a first lateral flow test strip, a second lateral flow test strip, and a dosing area, the dosing area interconnected with the first and second lateral flow test strips. The system further includes a collector for collecting a sample and a first mixer for receiving the sample from the collector, the mixer including buffers for mixing with the sample, the first mixer for dosing the sample pad a first time. The first lateral flow test strip provides for the detection of LDL-C, and the second lateral flow test strip provides for the detection of LDL-P.

### CROSS REFERENCE TO RELATED APPLICATION

This application claims the benefit of U.S. Provisional Patent Application No. 62/234,527 filed Sep. 29, 2015, and hereby incorporated by reference to the same extent as though fully disclosed herein.

### BACKGROUND

Heart disease is a leading cause of death in today's society. The monitoring of blood analytes to assist in the monitoring and prediction of heart disease is frequently conducted by medical personnel. In many scenarios, a blood sample must be taken far in advance of meeting with medical personnel so that laboratory testing may occur. Furthermore, recent developments in research related to heart disease have determined that certain blood analytes may be more relevant to the prediction and monitoring of heart disease than others. Typically, low-density lipoprotein is thought to cause cardiovascular disease by transporting cholesterol to the artery wall. Studies exist showing the cholesterol carrying capacity of such molecules. However, it has been determined that LDL cholesterol (LDL-C) is only one property of LDL related particles. LDL particles (LDL-P) also affect the progression of cardiovascular disease. In fact, in some scenarios, it has been determined that the measurement of LDL-P is more instructive in determining heart disease risk than measuring LDL-C. Therefore, it would be desirable to provide a point-of-care test that would test for LDL-C and possibly LDL-P simultaneously.

### BRIEF SUMMARY

In one embodiment, a system for testing for LDL-C and LDL-P includes a first lateral flow test strip, a second lateral flow test strip, and a dosing area, the dosing area interconnected with the first and second lateral flow test strips. The system further includes a collector for collecting a sample and a first mixer for receiving the sample from the collector, the mixer including buffers for mixing with the sample, the first mixer for dosing the sample pad a first time. The first lateral flow test strip provides for the detection of LDL-P, and the second lateral flow test strip provides for the detection of LDL-C. In one alternative, the system further includes a second mixer, the second mixer containing an enzyme formulation, the enzyme formulation for dosing the sample pad. Optionally, the first lateral flow test strip includes an antibody-antigen stripe and an antigen stripe. Alternatively, the antibody-antigen stripe is an anti Apo B Ab-latex conjugate. In one alternative, the antibody-antigen stripe includes blue particles connected to the anti Apo B-100 antibody. Optionally, the antigen stripe is an Apo B-100 antigen stripe, located in a direction of flow distal from the dosing area on the first lateral flow test strip. Alternatively, the second lateral flow test strip includes an anti Apo B-100 antibody stripe. In one configuration, the first and second lateral flow test strips include conjugate nitrocellulose and nylon membranes.

In one embodiment, a method of testing for LDL-C and LDL-P includes providing a meter, a first lateral flow test strip, a second lateral flow test strip, and a dosing area, the dosing area interconnected with the first and second lateral flow test strips, a collector for collecting a sample, and a first mixer. The method further includes collecting a sample with the collector and mixing the sample with the mixer. The method further includes dosing the sample on the dosing area. The method further includes laterally flowing the sample across the first and second lateral flow test strips and reading the first lateral flow test strip to determine a concentration of LDL-P in the sample. In one alternative, the method further includes providing a sampler, the sampler containing an enzyme formula, dosing the dosing area with the enzyme formula, and reading the second lateral flow test strip with the meter to determine a concentration of LDL-C in the sample. In another alternative, the first lateral flow test strip includes an antibody-antigen stripe and an antigen stripe. Optionally, the antibody-antigen stripe is an anti Apo B Ab-latex conjugate. Alternatively, the antibody-antigen stripe includes blue particles connected to the anti Apo B-100 antibody. Optionally, the antigen stripe is an Apo B-100 antigen stripe, located in a direction of flow distal from the dosing area on the first lateral flow test strip. In another alternative, the second lateral flow test strip includes an anti Apo B-100 antibody stripe. Alternatively, the first and second lateral flow test strips include conjugate nitrocellulose and nylon membranes. Optionally, the method further includes, after the dosing of the sample, flowing the sample to the antibody-antigen stripe of the first lateral flow test strip; binding the anti Apo B-100 antibody to LDL-P in the sample; flowing the sample to the antigen stripe of the first lateral flow test strip; and capturing unbound portions of the anti Apo B-100 antibody to the antigen stripe. Alternatively, the method further includes, after the dosing of the sample, flowing the sample to the anti Apo B-100 antibody stripe of the second lateral flow test strip; and binding LDL to the anti Apo B-100 antibody stripe of the second lateral flow test strip. Alternatively, the method further includes, after the dosing with the enzyme formula, flowing the enzyme formula to bound LDL, the bound LDL being a result of the binding LDL to the anti Apo B-100 antibody stripe; and reacting the enzyme formula with the bound LDL.

### BRIEF DESCRIPTION OF THE DRAWINGS

FIG. 1 shows one embodiment of a lateral flow test strip;

FIG. 2 shows one embodiment of a first lateral flow test strip and a second lateral flow test strip for use in the detection LDL-C and LDL-P;

FIG. 3 shows one embodiment of a sample collector for use with the first and second test strips for use in the detection of LDL-C and LDL-P;

FIG. 4 shows a sample collector (S1) with a mixer containing reagents for use with the first and second test strips for use in the detection of LDL-C and LDL-P;

FIGS. 5a and 5b show a second sampler (S2) containing a proprietary enzymatic formulary for use with the first and second test strips for use in the detection LDL-C and LDL-P;

FIG. 6 shows a typical dose response of Apo B-100 particle concentration to the referenced method; and

FIG. 7 shows a typical dose response of LDL cholesterol concentration to the referenced method.

### DETAILED DESCRIPTION

Certain terminology is used herein for convenience only and is not to be taken as a limitation on the embodiments of the systems and methods for point-of-care determination of LDL-C and LDL-P. In the drawings, the same reference letters are employed for designating the same elements throughout the several figures.

Currently, there are no point-of-care (POC) methods to determine the LDL particle (LDL-P) concentration of LDL cholesterol (LDL-C) simultaneously in a whole blood sample. Embodiments of systems and methods for point-of-care determination of LDL-C and LDL-P determine the number of LDL particles (LDL-P) and LDL cholesterol (LDL-C) concentrations. Embodiments of systems and methods for point-of-care determination of LDL-C and LDL-P yield an LDL-P (particle) concentration and a direct LDL-C (cholesterol) concentration from a single sample using a single device. This point-of-care device test (POCT) can give both particle and cholesterol concentrations. In recent literature, it has been recognized that LDL-P concentration is of greater diagnostic value. In addition, it also is important to provide actual LDL-C concentrations in the lipoprotein fractions. Embodiments of systems and methods for point-of-care determination of LDL-C and LDL-P include a POCT for determining both the LDL-P (particle) and LDL-C (cholesterol) concentrations in one simple test.

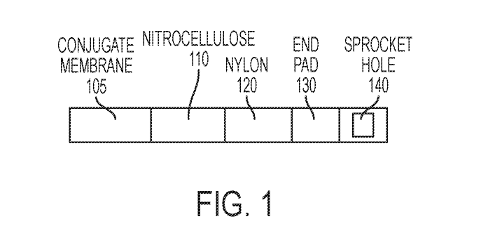

Embodiments of systems and methods for point-of-care determination of LDL-C and LDL-P include a POCT that employs a lateral flow methodology. FIG. 1 shows one embodiment of a lateral flow test strip that may be used in conjunction with the systems and methods described herein. In lateral flow, the conjugate membrane 105, nitrocellulose 110, and nylon 120 membranes are layered in such a way to obtain easy plasma/fluid flow which enables the analytes to be captured on the membranes in different zones as shown in FIG. 1. The flow is towards end pad 130, where the concentration of analytes is read. Additionally, the lateral flow test strip includes a sprocket hole 140 for mounting and positioning the strip in a cassette or other holder. This lateral flow based device has two parts: the left arm of the lateral flow which will quantify the concentration of Apo B-100 concentration, and the right arm of the device which will quantify the cholesterol concentration present in the LDL's Apo B-100 fractions. The resulting device will give physicians a dual value to make effective diagnoses or treatment options for patients with a hypercholesterolemic condition.

### Description of Quantification of LDL-P

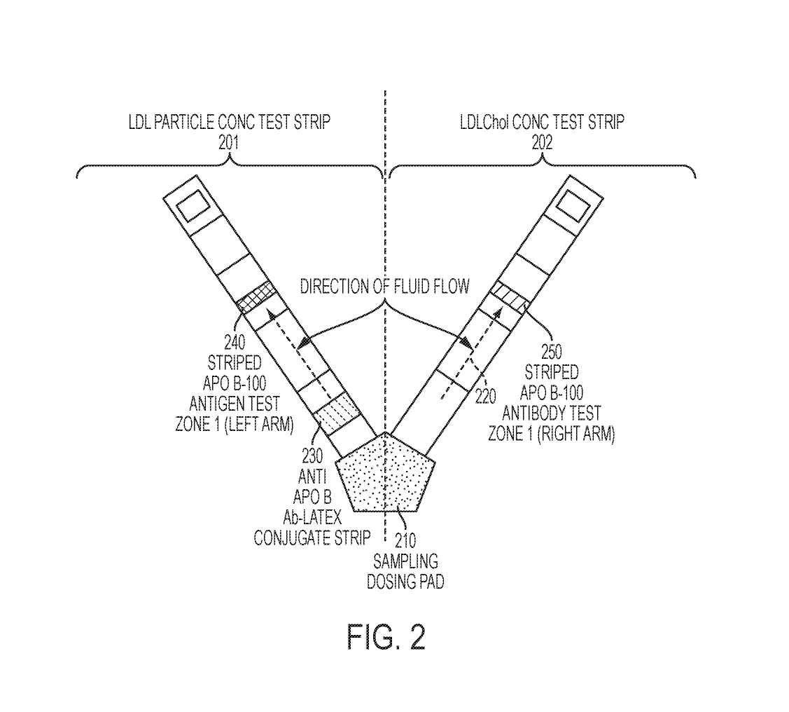

As shown in FIG. 2, embodiments of a device for quantification of LDL-P and LDL-C include two arms 201, 202. Each arm 201, 202 is a lateral flow test strip with a common sample dosing pad 210. In this embodiment, the LDL-P arm 201, a lateral flow method using an antibody-antigen interaction to quantify the levels of LDL's Apo B-100 protein in the sample is utilized. An antibody-antigen stripe 230 (anti Apo B Ab-latex conjugate stripe) is included in the lateral flow strip. Blue particles (or alternatively other detectable particle colors) coated with anti Apo B-100 antibody will be striped as shown in FIG. 2 at stripe 230. The Apo B-100 antigen will be striped in zone one at stripe 240.

### Description of Quantification of LDL-C

In the second arm, arm 202, a lateral flow method using an enzymatic reaction of the LDL's fraction in the sample will be quantified. Here, only the anti Apo B-100 antibody will be striped in zone one at stripe 250. No blue dye particles will be used. As can be seen in the figures, the fluid flows from the dosing pad 210 toward the other end of the lateral flow strip in the flow direction toward stripe 250.

As shown in FIG. 2, the architecture of the device includes arms 201, 202 where the LDL-P concentration and LDL-C concentration can be determined. The left arm 201 of the device yields an LDL-P concentration based on the lipoprotein Apo B-100 concentration, while the right arm 202 of the device yields an LDL-C concentration based on enzymatic reaction of the cholesterol present in the LDL Apo lipoprotein. FIG. 2 shows the locations of the striped components and the zones for each lipoprotein.

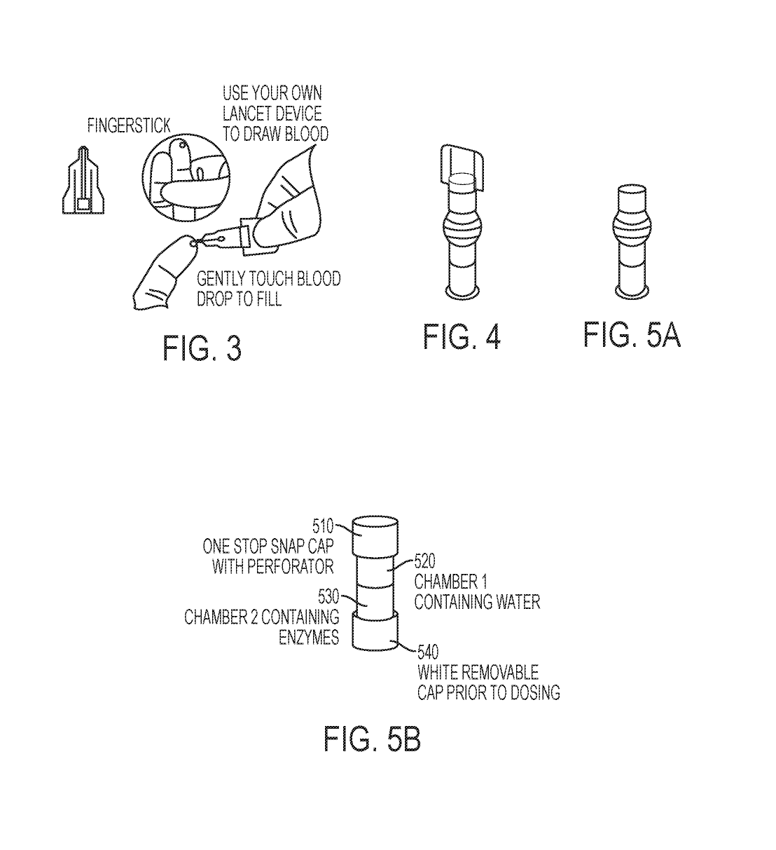

Dosing and quantification occur according to a number of steps. FIG. 3 shows a sample collector with whole blood and the method for collecting capillary whole blood from a finger stick. FIG. 4 shows a sample collector (S1) with a mixer containing reagents. FIGS. 5a and 5b show a second sampler (S2) containing proprietary enzymatic formulary. FIG. 5b shows that the second sampler includes a one-stop snap cap 510 with a perforator for perforating the chamber 520 containing water which may later be mixed with the chamber 530 containing enzymes and then dosed by removing the cap 540. p Step 1: A venous or capillary sample will be collected by the collector as shown in FIG. 3, mixed with proprietary buffers by sample mixers (S1) (as shown in FIG. 4), and dosed on the sample pad. After dosing, the solution will wick on both arms evenly. The following physical phenomenon will ensue.

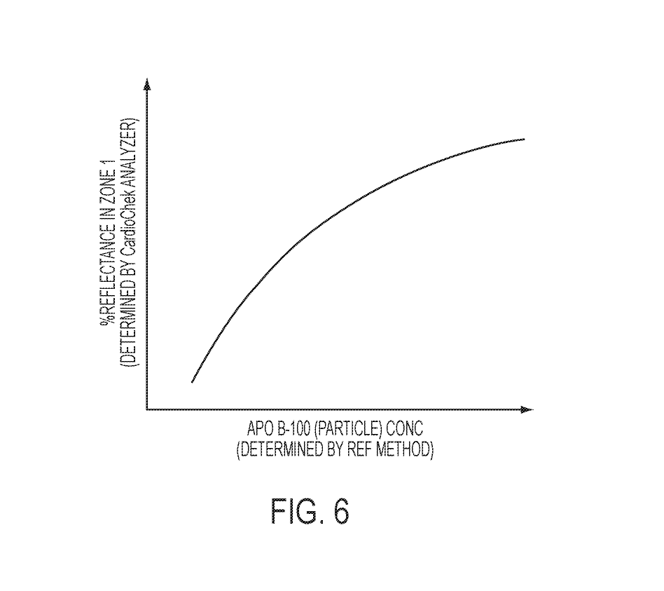

The blue particles in stripe 230 in the left arm 201 of the device will interact with the sample and migrate along the length of the lateral flow strip. The blue particles coated with anti Apo B-100 antibody will be captured in the test zone one at stripe 240 (which is striped with Apo B-100 antigen-protein conjugate) when a very small amount of LDL-P is present in the sample. If a large amount of LDL-P is present in the sample, the LDL will stick to the blue particles resulting in proportionally lower capture of the blue particles in zone one at stripe 240. In this particular immunochemistry method, a direct relationship exists between the analyte concentrations to the light reflected from the “capture” in zone one at stripe 240 (for both arms of the device) as shown in the dose response, FIG. 6. A lower reflectance reading will yield a lower value, while a high reflectance reading will yield a higher analyte concentration. An LDL-P can be calibrated (FIG. 6) against the Liposcience NMR® method or via the Roche's “Tina-quant Apo lipoprotein A-1 ver.2®” assay, thus enabling a POC device now to report the particle count for the LDL lipoproteins.

In the same amount of time, the LDL's Apo B-100 fractions will migrate on the right arm and will be captured on the zone one at stripe 250 where the anti-Apo B-100 will be striped, letting all other lipoproteins flow to the end pad and be ready for step 2.

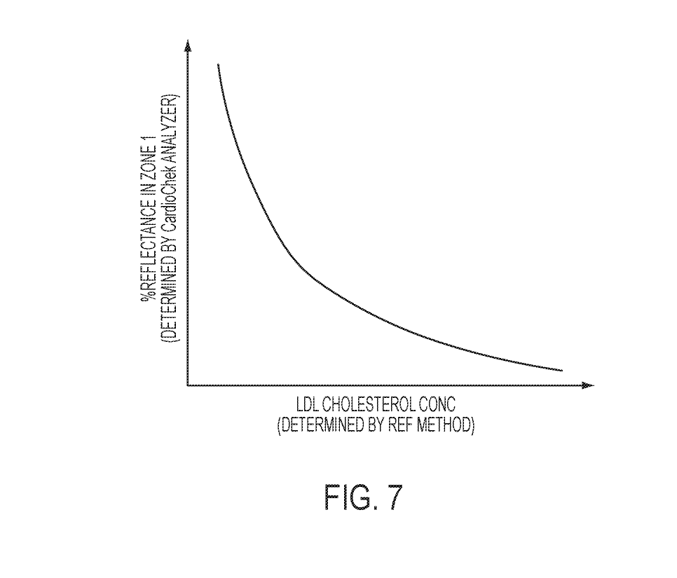

Step 2: When the LDL-P concentration has been determined (from the left arm strip) by the meter, the meter will prompt the user to dose a second sampler S2 (as shown in FIG. 5) containing a proprietary enzyme formulary. In this embodiment, the enzymes will wick to capture Apo B-100 fractions in zone one of the left arm and trigger a series of reactions where the cholesterol concentration for LDL will be determined (LDL-C). The enzymatic formulary contains surfactants to de-coat the Apo B-100 lipoprotein followed by a series of chemical reactions of the cholesterol esters by cholesterol esterase, cholesterol oxidase. The resulting hydrogen peroxide then will be reacted with 4-amino antipyrine (4-AAP) and N-Ethyl-N-(2-hydroxy-3-sulfopropyl)-3,5-dimethylaniline, sodium salt, monohydrate (MAOS) with the help of horse radish peroxidase (HRP) enzyme to yield a blue colored quinoneimine dye which will be measured by the meter in reflectance mode. This reaction scheme is merely an example of a reaction scheme that may be utilized in the development of a color change, and other reaction schemes may be apparent to those of ordinary skill in the art. In certain embodiments, an LDL-C is calibrated (FIG. 7) against the Liposcience NMR® method or via the Roche's “Tina-quant Apo lipoprotein B ver.2®” assay, thus enabling a POC device now to report the particle count for the LDL lipoproteins.

In one of the embodiments of step 2, the enzymes can be lyophilized and placed in a hand-held mixer that contains two compartments. Compartment one 530 is for lyophilized enzymes, while compartment two 520 is for holding the right amount of water (see FIG. 5b) for mixing the enzyme prior to dosing it. The water and the enzymes will be mixed by simply puncturing the barrier between them. FIG. 6 shows a typical dose response of Apo B-100 particle concentration to the referenced method. FIG. 7 shows a typical dose response of LDL cholesterol concentration to the referenced method.

While specific embodiments have been described in detail in the foregoing detailed description and illustrated in the accompanying drawings, it will be appreciated by those skilled in the art that various modifications and alternatives to those details could be developed in light of the overall teachings of the disclosure and the broad inventive concepts thereof. It is understood, therefore, that the scope of this disclosure is not limited to the particular examples and implementations disclosed herein but is intended to cover modifications within the spirit and scope thereof as defined by the appended claims and any and all equivalents thereof. Note that, although particular embodiments are shown, features of each may be interchanged between embodiments.

What is claimed as new and desired to be protected by Letters Patent of the United States is:

1. A system for testing for LDL-C and LDL-P, the system comprising:
a first lateral flow test strip and a second lateral flow test strip;
a dosing area, the dosing area interconnected with the first and second lateral flow test strips;
a collector for collecting a sample; and
a first mixer for receiving the sample from the collector, the mixer including buffers for mixing with the sample, the first mixer for dosing the sample pad a first time, wherein the first lateral flow test strip provides for the detection of LDL-C and the second lateral flow test strip provides for the detection of LDL-P.

2. The system of claim 1, further comprising:
a second mixer, the second mixer containing an enzyme formulation, the enzyme formulation for dosing the sample pad.

3. The system of claim 1, wherein the first lateral flow test strip includes an antibody-antigen stripe and an antigen stripe.

4. The system of claim 3, wherein the antibody-antigen stripe is an anti Apo B Ab-latex conjugate.

5. The system of claim 3, wherein the antibody-antigen stripe includes blue particles connected to the anti Apo B-100 antibody.

6. The system of claim 4, wherein the antigen stripe is an Apo B-100 antigen stripe, located in a direction of flow distal from the dosing area on the first lateral flow test strip.

7. The system of claim 6, wherein the second lateral flow test strip includes an anti Apo B-100 antibody stripe.

8. The system of claim 1, wherein the first and second lateral flow test strips include conjugate, nitrocellulose, and nylon membranes.

9. A method of testing for LDL-C and LDL-P, the method comprising:
providing a meter, a first lateral flow test strip, a second lateral flow test strip, a dosing area, the dosing area interconnected with the first and second lateral flow test strips, a collector for collecting a sample, and a first mixer;
collecting a sample with the collector;
mixing the sample with the mixer;
dosing the sample on the dosing area;
laterally flowing the sample across the first and second lateral flow test strips; and
reading the first lateral flow test strip to determine a concentration of LDL-P in the sample.

10. The method of claim 9, further comprising:
providing a sampler, the sampler containing an enzyme formula;
dosing the dosing area with the enzyme formula; and
reading the second lateral flow test strip with the meter to determine a concentration of LDL-C in the sample.

11. The method of claim 10, wherein the first lateral flow test strip includes an antibody-antigen stripe and an antigen stripe.

12. The method of claim 11, wherein the antibody-antigen stripe is an anti Apo B Ab-latex conjugate.

13. The method of claim 11, wherein the antibody-antigen stripe includes blue particles connected to the anti Apo B-100 antibody.

14. The method of claim 12, wherein the antigen stripe is an Apo B-100 antigen stripe, located in a direction of flow distal from the dosing area on the first lateral flow test strip.

15. The method of claim 14, wherein the second lateral flow test strip includes an anti Apo B-100 antibody stripe.

16. The method of claim 15, wherein the first and second lateral flow test strips include conjugate, nitrocellulose, and nylon membranes.

17. The method of claim 15, further comprising, after the dosing of the sample:
flowing the sample to the antibody-antigen stripe of the first lateral flow test strip;
binding the anti Apo B-100 antibody to the LDL-P in the sample;
flowing the sample to the antigen stripe of the first lateral flow test strip; and
capturing unbound portions of the anti Apo B-100 antibody to the antigen stripe.

18. The method of claim 17, further comprising, after the dosing of the sample:
flowing the sample to the anti Apo B-100 antibody stripe of the second lateral flow test strip; and
binding LDL to the anti Apo B-100 antibody stripe of the second lateral flow test strip.

19. The method of claim 18, further comprising, after the dosing with the enzyme formula:
flowing the enzyme formula to bound LDL, the bound LDL being a result of binding LDL to the anti Apo B-100 antibody stripe; and
reacting the enzyme formula with the bound LDL.

20. The method of claim 19, wherein the reading of the first lateral flow test strip and reading of the second lateral flow test strip include reading an optical property with the meter for each reading.
